(19) United States
(12) Patent Application Publication
(10) Pub. No.: US 2009/0156967 A1
(43) Pub. Date: Jun. 18, 2009

(54) System for evaluating an infant's perception of aroma

(75) Inventor: Jason C. Cohen, Appleton, WI (US)

Correspondence Address: KIMBERLY-CLARK WORLDWIDE, INC.;Catherine E. Wolf, 401 NORTH LAKE STREET, NEENAH, WI, 54956, US

(73) Assignee: Kimberly-Clark Worldwide, Inc.

(21) Appl. No.: 12/002,033

(22) Filed: Dec. 14, 2007

(51) Int. Cl. A61B 5/11; G01D 21/00; A61B 5/00

(52) U.S. Cl. 600/590; 73/865.4; 600/300

(57) ABSTRACT

Disclosed is a system, method, and device for detecting non-nutritive sucking events produced by an infant; patterns comprising such events (i.e., rhythmic sucking patterns, or RSPs); and relating said events and/or RSPs to an infant's response to a olfactory sensory input. The frequency and/or amplitude and/or other characteristics of non-nutritive sucking can change in response to such inputs. Accordingly, changes to RSPs offer detectable and measurable indicia of an infant's perception of these inputs. Furthermore, such information may be used to support and/or substantiate marketing communications to consumers; to guide selection and/or management of research-and-development projects; to help choose product designs that elicit RSPs identified as correlating with the well being of an infant; and other such uses.

### BACKGROUND

The sense of smell is an important part of most people's every day life. Aromas can help evoke an emotion, a memory, an object, a person, etc. Or an aroma can simply be pleasing in and of itself. Accordingly, businesses seek to sell or employ aromas that help meet consumers' perceived needs. Businesses do this, in part, by spending money and time researching and developing aromas, and products employing aromas. Businesses may evaluate aromas by conducting use tests. In these kinds of tests, participants are asked to use products employing one or more aromas, or simply to evaluate the aromas themselves. After the test is over, participants answer questions about the product, aroma(s), or both. Businesses may evaluate other variables in addition to aroma in such tests. For example, a manufacturer might evaluate the effect of color and aroma on use-test participants' perception of various qualitative aspects of a product that's being evaluated. A manufacturer can use various experimental designs when conducting such use tests to evaluate the effect of one or more independent variables on a dependent variable of interest. When the results of such tests are to be used to support an advertising claim, a manufacturer will typically work to ensure that the test and its interpretation comport with applicable guidelines set out in standardized test methods (e.g., ASTM guidelines for product testing), or with accepted practices within a given industry.

Unfortunately, an infant's perception of a product employing an aroma may be difficult to determine and/or quantify. Generally, an infant provides signals as to his or her comfort, well being, or stress level in the form of facial expressions, other physical motions, and sounds. Some signals, such as smiling and cooing, may indicate that the infant is satisfied or happy with current environmental conditions and/or bodily conditions. Other signals, such as crying, generally indicate that the infant is not satisfied with current environmental conditions and/or bodily conditions. Such signals, whether indicative of a positive state of well being (smiling and cooing) or a negative state of well being (crying) may not be quantifiable and/or readily correlatable to the infant's perception of a product's performance (e.g., the perception of a product employing an aroma).

What is needed is a system, device, and method by which indicia of an infant's perception of an aroma, or product employing an aroma, is sensed, quantified, and used to help evaluate the performance of said aroma, product, or both. The information obtained with such a system could be used for a variety of purposes, including: comparing a plurality of products employing one or more aromas; comparing one or more aromas; comparing intensities or concentrations of one or more aromas, or products employing aromas of different intensities or concentrations; changing the functional performance and/or technical specifications and/or materials-of-construction of a product in response to the obtained information; communicating messages to consumers regarding the performance of a product based on the obtained information; and conducting, or making decisions with respect to, other research-and-development and/or marketing activities.

### SUMMARY

We have determined that non-nutritive sucking events produced by an infant, and patterns comprising such events (i.e., rhythmic sucking patterns), can be used to help evaluate an infant's perception of an aroma, its intensity or concentration, or a product employing an aroma. A rhythmic sucking pattern comprises a pattern of individual, non-nutritive, sucking events as evidenced by, for example, an infant sucking on a pacifier. We have confirmed that the frequency and/or amplitude and/or other characteristics of non-nutritive sucking events change in response to a change in aroma (whether by its introduction, absence, or change in concentration/intensity). Thus rhythmic sucking patterns, and changes to rhythmic sucking patterns, offer a detectable and measurable signal by which an infant's perception of an aroma, or its absence, may be characterized.

One version of the invention is a system for evaluating an infant's perception of an aroma, the system comprising: a sensor adapted to detect non-nutritive sucking events produced by an infant; a source comprising an aroma adapted to be released from the source; and an information device operatively connected to the sensor, said information device adapted to accomplish one or more of receiving, storing, processing, displaying, or transmitting information corresponding to non-nutritive sucking events detected by the sensor.

In other versions of the invention, the sensor is a pressure transducer adapted to detect non-nutritive sucking events. The pressure transducer may or may not be attached directly to the pacifier itself.

In some versions of the invention, the information device is attached directly to the pacifier.

In some representative embodiments of the invention, the information device is also operatively connected to the source, said information device further adapted to accomplish one or more of receiving, storing, processing, displaying, or transmitting information corresponding to the source's release of the aroma (or other change to the presence of an aroma, such as an increase or decrease in the amount/concentration of the aroma, or chemicals or molecules corresponding to the aroma).

In other versions of the invention, the aforementioned system further comprises a second information device that is operatively connected to the source, said second information device adapted to accomplish one or more of receiving, storing, processing, displaying, or transmitting information corresponding to the source's release of the aroma (or other change to the presence of an aroma, such as an increase or decrease in the amount/concentration of the aroma, or chemicals or molecules corresponding to the aroma).

The information device and/or the second information device may be operatively connected to the sensor, source, or the combination of the two by conductive wire, a pneumatic connection, a hydraulic connection, a connection using a portion of the electromagnetic spectrum, or some combination of these.

One version of a method for evaluating an infant's response to a first aroma comprises the steps of: (a) detecting non-nutritive sucking events produced by an infant; and (b) relating the detected non-nutritive sucking events to a change in a first aroma proximate to the infant. Change encompasses the presence or absence of an aroma. Change also encompasses an increase or decrease in the intensity or concentration of the aroma.

Another version of a method for evaluating one or more infants' responses to one or more aromas comprises the steps of: repeating steps (a) and (b) as described in the immediately preceding paragraph for a second aroma; and comparing the detected non-nutritive sucking events produced by an infant proximate to the change in the first aroma to the detected non-nutritive sucking events produced by an infant proximate to the change in the second aroma. Comparing a first and second aroma encompasses comparing two different aromas, comparing two different concentrations or intensities of one aroma, or comparing different intensities or concentrations of two different aromas.

Other methods of the present invention comprise communicating a message based on the relationship between detected non-nutritive sucking events produced by an infant and any change to a first aroma proximate to the infant (e.g., by introducing an aroma where none was present before; by changing the concentration of an aroma; by eliminating an aroma, as by, for example, adsorbing or absorbing one or more chemical components corresponding to the aroma on a solid, such as activated carbon, or a liquid; etc.).

Another representative method of the present invention comprises communicating a message based on the comparison between the detected non-nutritive sucking events produced by an infant proximate to the change in the first aroma to the detected non-nutritive sucking events produced by an infant proximate to the change in the second aroma.

Another version of the present invention is a message adapted to be communicated, wherein the message comprises information produced using one of the systems of the present invention.

Another version of the present invention is a message adapted to be communicated and referring to an aroma, wherein substantiation of the message is based on non-nutritive sucking events. The term “substantiation” has the definition generally ascribed to it in advertising law, viz., that there is a reasonable basis in fact for the message's content (except for content deemed to be hyperbole or puffery, in which case a reasonable basis in fact need not be present when presenting such hyperbole or puffery). Where applicable, “substantiation” may implicate standard test methods that help guide manufacturers on the nature of tests and statistical analyses used to support advertising claims (e.g., ASTM E 1958-06, entitled “Standard Guide for Sensory Claim Substantiation”).

Another version of the invention is a research-and-development project directed to selecting an aroma, wherein said research-and-development project comprises an evaluation of non-nutritive sucking events and relating these events to the presence, absence, or change in one or more aromas. A manufacturer or seller may conduct a program of this type to select an aroma for sale, either alone or as employed in a product (e.g., a disposable washcloth; a stuffed animal; a lotion; a soap, whether in solid or liquid form; a shampoo; an ingestible product, such as a condiment, tablet, or liquid; and any other product in which an aroma might be employed).

Another version of the invention is a system for evaluating an infant's perception of an aroma, the system comprising a first sensor adapted to detect non-nutritive sucking events produced by an infant; a second sensor adapted to detect a property characterizing the presence of an aroma proximate to the infant; and an information device operatively connected to the first sensor, said information device adapted to accomplish one or more of receiving, storing, processing, displaying, or transmitting information corresponding to the non-nutritive sucking events detected by the first sensor.

In another embodiment, the second sensor of the aforementioned system is adapted to detect a property indicative of a change to the presence of an aroma in the environment proximate to the infant perceiving the aroma (whether the aroma is being released, changed in some way—as would occur if its intensity or concentration is being changed, or removed—as would occur by absorbing or adsorbing one or more of the chemical components on a solid surface such as activated carbon, and the like). Examples of such properties include: mass flow rate of a chemical corresponding to an aroma to be released; volumetric flow rate of a chemical corresponding to an aroma to be released; mass of a chemical corresponding to an aroma to be released; volume of a chemical corresponding to an aroma to be released; and the like; or some combination of these.

In some versions of the aforementioned system, the information device that is operatively connected to the first sensor is also operatively connected to the second sensor. Accordingly, said information device is further adapted to accomplish one or more of receiving, storing, processing, displaying, or transmitting information corresponding to the second sensor's detection of a property corresponding to a change to the presence of an aroma in the environment proximate to the infant perceiving the aroma.

In other versions of the invention, the aforementioned system comprises a second information device that is operatively connected to the second sensor. The second information device is adapted to accomplish one or more of receiving, storing, processing, displaying, or transmitting information corresponding to the second sensor's detection of a property corresponding to a change to the presence of an aroma in the environment proximate to the infant perceiving the aroma.

Another version of the invention is a method for evaluating an infant's perception of an aroma, the method comprising: (a) detecting non-nutritive sucking events produced by an infant proximate to a change to an aroma; (b) characterizing the change to the aroma; and (c) relating the detected non-nutritive sucking events to the characterized change to the aroma.

In some representative versions of a method for evaluating an infant's perception of aroma, the non-nutritive sucking events are detected, at least in part, while the infant is in an environment substantially devoid of sounds. This might be done to minimize sonic stimuli that may confound the results of the evaluation of an infant's perception of aroma. So, for example, the infant may be placed in a quiet room, or other environment that is substantially acoustically isolated. To further enhance such isolation, a pacifier adapted to wirelessly transmit data corresponding to the detected non-nutritive sucking events to a remote information device, such as a computer, may be employed. In this way the computer, as well as human activity surrounding operation of the computer, can be located in a room separate from the environment in which the infant is placed during the evaluation. Alternatively, the infant participant may be in the same room as the researcher or other personnel, along with equipment used to monitor the non-nutritive sucking. For example, the infant might be placed in one end of the room, facing a wall, with research personnel and equipment at the opposite end of the room, with the personnel and equipment not visible to the infant.

To further limit confounding effects, a plurality of pacifier models may be made available, with one pacifier model being selected for conducting the evaluation. In some versions of the invention, the infant's caregiver will select the pacifier model to be employed during the evaluation. Typically the selected pacifier model will correspond to a pacifier model that is generally used by the infant apart from the evaluation. Each of these pacifier models may be made so that they are able to wirelessly transmit data, thereby avoiding wires or other physical connections between the pacifier and an information device used to monitor the evaluation (for those embodiments in which the information device is separate from, and physically connected to, the pacifier). These wires could prove distracting to the infant (e.g., because the wires drape over the infant, or are visually distracting, or tug on the pacifier in some way—potentially affecting infant's rhythmic sucking pattern).

In another version of a method for evaluating an infant's response to a change in an aroma, the evaluation is conducted while the infant is sleeping. Such an evaluation should, of course, limit unintended sensory stimuli that might be perceived by the infant during the test (e.g., perceiving movement, color, or other visual stimuli; perceiving sounds; etc.).

Another method for evaluating an infant's perception of a product attribute comprises the steps of: (a) providing a plurality of pacifier models adapted to detect non-nutritive sucking events and wirelessly transmit data corresponding to the detected non-nutritive sucking events to a remote information device; (b) selecting one of the pacifier models for the evaluation; (c) using the selected pacifier model to detect non-nutritive sucking events produced by an infant who is exposed to a sensory stimulus that corresponds to the product attribute; (d) relating the detected non-nutritive sucking events to the sensory stimulus, the product attribute, or both.

In other versions of a method for evaluating an infant's perception of a product attribute, the non-nutritive sucking events are detected, at least in part, while the infant is in an environment substantially devoid of sounds. This might be done to minimize sonic stimuli that may confound the results of the evaluation of an infant's perception of the sensory stimulus and/or product attribute of interest. So, for example, the infant may be placed in a quiet room, or other environment that is substantially acoustically isolated.

In some versions of the invention, the infant's caregiver will select the pacifier model to be employed during the evaluation. Typically the selected pacifier model will correspond to, or substantially match, a pacifier model that is generally used by the infant apart from the evaluation.

In other versions of a method for evaluating an infant's perception of a product attribute, the evaluation is conducted while the infant is sleeping. Such an evaluation should, of course, limit unintended sensory stimuli that might be perceived by the infant during the test (e.g., perceiving movement, color, or other visual stimuli; perceiving sounds; etc.).

In other versions of a method for evaluating an infant's perception of a product attribute, the remote information device displays data corresponding to the non-nutritive sucking events detected by the selected pacifier model.

In other versions of a method for evaluating an infant's perception of a product attribute, the remote information device employs a graphical user interface adapted to display time; the amplitude and/or the frequency of the detected non-nutritive sucking events; data corresponding to the identity and/or other characteristics of the infant participating in the evaluation; and data corresponding to the sensory stimulus and/or product attribute being evaluated.

These and other versions, embodiments, and examples of the invention are discussed elsewhere in this application.

### DRAWINGS

FIG. 1 representatively illustrates a pacifier adapted to detect non-nutritive sucking events produced by a baby sucking on the pacifier.

FIGS. 2A, 2B, 2C, 26D, 2E, and 2F representatively depict modification of a pacifier into one version of a device adapted to detect non-nutritive sucking events and/or rhythmic sucking patterns.

FIG. 3 representatively depicts modification of a pacifier into one version of a device adapted to detect non-nutritive sucking events and/or rhythmic sucking patterns.

FIGS. 4A and 4B representatively depict modification of a pacifier into one version of a device adapted to detect non-nutritive sucking events and/or rhythmic sucking patterns.

FIGS. 5A and 5B representatively depict another version of a device adapted to detect non-nutritive sucking events and/or rhythmic sucking patterns.

FIG. 6 representatively depicts the interconnected components of one version of a device adapted to detect non-nutritive sucking events and/or rhythmic sucking patterns.

FIG. 7 representatively depicts a version of a system for detecting both non-nutritive sucking events/rhythmic sucking patterns and a change to a property corresponding to an aroma proximate to an infant.

Corresponding reference characters indicate corresponding parts throughout the drawings.

### DEFINITIONS

Within the context of this specification, each term or phrase below includes the following meaning or meanings:

The terms “disposed on,” “disposed along,” “disposed with,” or “disposed toward” and variations thereof are intended to mean that one element can be integral with another element, or that one element can be a separate structure bonded to or placed with or placed near another element.

“Graphical user interface” refers to a visual display, such as that found on a monitor, computer display, hand-held computer or personal-digital assistant, or other such information device, and includes the ways by which a user sees and manipulates information displayed via the interface. For example, a graphical user interface may include a visual representation of an x-y chart, with the x dimension corresponding to time and the y dimension corresponding to pressure or other parameter that correlates with non-nutritive sucking events. The visual representation can be updated periodically so that the visual representation presents a real-time display of the non-nutritive sucking events detected by a pacifier adapted to detect such events.

“Operatively connected” refers to the communication pathway by which one element, such as a sensor, communicates with another element, such as an information device. Communication may occur by way of an electrical connection through a conductive wire. Or communication may occur via a transmitted signal such as an infrared frequency, a radio frequency, or some other transmitted frequency signal. Alternatively, communication may occur by way of a mechanical connection, such as a hydraulic or pneumatic connection.

These terms may be defined with additional language in the remaining portions of the specification.

### DESCRIPTION

### Representative Devices and Sensors for Detecting Non-Nutritive Sucking Events

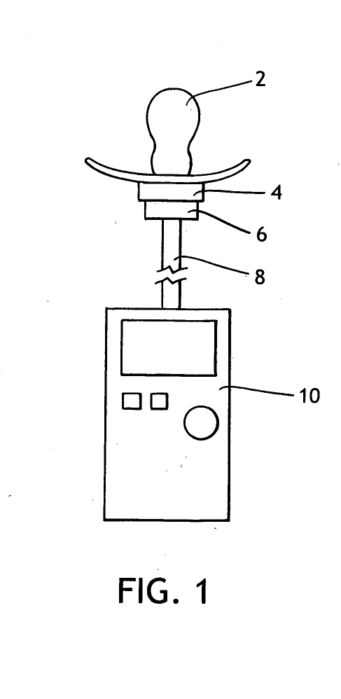

A representative device, adapted to detect non-nutritive sucking events produced by an infant, is depicted in FIG. 1, along with an information device. A nipple 2 is attached to a base 4. A sensor 6 is attached to the base so that an infant's sucking on the nipple is detected. For example, as outlined in the Examples section below, we modified commercially available pacifiers (e.g., a NUK® brand pacifier made by NUK, MAPA GmbH, Industriestrasse 21-25, D-27404, Zeven, Germany; a MAM® brand pacifier made by MAM Babyartikel GEsmbh, Lorenz-Mandl-Gasse 50, 1160 Wien, Austria; a Disney® brand/The-first-years®-brand pacifier made by The First Years, One Kiddie Drive, Avon, Mass.; and a Playtex® brand pacifier, made by Playtex®, a business having offices in Allendale, N.J.) by attaching a pressure transducer to the base of each pacifier using an epoxy glue. For a number of pacifiers (e.g., those described in Examples 1-3 below), we used pressure transducers available from Omega Engineering, having offices at One Omega Drive, Box 4047, Stamford, Conn. As discussed below in Examples 1-3, three different pressure transducers, each capable of measuring different ranges of pressure, were used: (1) 0 to 1 pound per square inch (PSI) (model no. PX26-001GV, which corresponds to 0 to about 16.7 millivolts DC full scale); (2) 0 to 5 PSI (model no. PX26-005GV, which corresponds to 0 to about 50 millivolts DC full scale); and (3) 0 to 15 PSI (model no. PX26-015GV, which corresponds to 0 to about 100 millivolts DC full scale). An epoxy adhesive available from Cole-Parmer Instrument Company, having offices at 625 East Bunker Court, Vernon Hills, Ill., was used to attach the pressure transducer to the base of the pacifier. By operatively connecting the modified pacifier to a digital multimeter available from Fluke Corporation, having offices at 6920 Seaway Boulevard, Everett, Wash. (or, as described below, a computer having an analog-to-digital device), we were able to monitor changes in pressure inside the nipple of the modified pacifier. In FIG. 1, the operative connection is exemplified by a wire 8 connected to an information device 10, in this case the aforementioned multimeter. As mentioned elsewhere, the sensor 6 may be operatively connected to an information device in various ways, including use of a conductive wire, a selected portion of the electromagnetic spectrum (e.g., a wireless connection using radio waves), or a mechanical connection (e.g., a pneumatic connection).

As described in Example 4 below, we have also made pacifiers in which a pressure transducer and wireless transmitter are attached to the base of various pacifier models. As with the pacifiers discussed above, the pressure transducer is in fluid communication with the air within the nipple so that, as described below, changes in pressure due to an infant sucking on the pacifier are detected and wirelessly transmitted to an information device, such as a computer. A representative system for wirelessly monitoring non-nutritive sucking events and corresponding rhythmic sucking patterns effected by an infant is described in Example 6 below.

An infant's sucking on the nipple causes the flexible nipple to stretch or extend, and then return to its original shape. This periodic extending or stretching of the nipple subjects the inside of the nipple to periodic compression, thereby changing the pressure inside the nipple. By operatively connecting a pressure transducer to the volume inside the nipple, the pressure inside the nipple, or a value corresponding to pressure inside the nipple, can be monitored. In some instances, we used a multimeter to display a reading, in millivolts, that corresponded to the pressure inside the nipple. In other cases we used a computer with an analog-to-digital device, and software adapted configure the computer for collecting and processing data, to process and display readings corresponding to the pressure inside the nipple. Additional detail regarding the construction of representative pacifiers adapted to detect non-nutritive sucking events and corresponding rhythmic sucking patterns, and representative versions of systems and other contexts employing such pacifiers, are given elsewhere in the present application, and in the Examples section below.

Other sensors may be used to detect non-nutritive sucking events produced by an infant. For example, a strain gauge could be attached to a pacifier to detect any deflection or deformation of one or more elements of the pacifier (e.g., the nipple; the base to which the nipple is attached; etc.).

Alternatively, a sensor for detecting electrical signals associated with contraction of a muscle or muscle group could be used to detect non-nutritive sucking events produced by an infant. For example, a sensor comprising electrodes and capable of being adhered to skin could be used to detect such sucking events, whether effected by an infant sucking on a pacifier, the nipple on a bottle, the infant's own thumb, finger, or fingers, and the like.

If the non-nutritive sucking events produce sounds, then a device for monitoring audible events could be used to detect the sounds corresponding to non-nutritive sucking events.

While the preceding paragraphs provide examples of devices, sensors, and methods that may be used to detect non-nutritive sucking events produced by an infant, as well as different ways by which infants effect non-nutritive sucking events (e.g., by sucking on a pacifier, a nipple on a bottle, the infant's own thumb, etc.), other devices, sensors, methods, and ways of generating non-nutritive sucking events may be used, so long as the selected approach is capable of detecting the non-nutritive sucking events.

### Representative Information Devices

A multimeter, discussed in the preceding paragraphs and in the Examples section below, is one version of an information device; i.e., a device adapted to accomplish one or more of receiving, storing, processing, displaying, or transmitting information, in this case information corresponding to non-nutritive sucking events produced by an infant, and detected by a sensor. For our work, the multimeter was used in some cases to display a reading, in millivolts, that corresponded to the pressure inside a modified pacifier. An infant's sucking on that pacifier produced a measurable rhythmic sucking pattern (“RSP”)—i.e., a series of millivolt readings that we could plot and evaluate.

A variety of information devices may be used in conjunction with the present invention. For example, a computer may be used to monitor one or more values corresponding to the non-nutritive sucking events produced by an infant. Generally, a computer is capable of receiving, storing, processing, displaying, and transmitting information. Through the use of appropriate software, the computer can be configured to receive, store, process, display, and/or transmit information corresponding to non-nutritive sucking events produced by an infant. In our work, we used a computer to accumulate individual millivolt readings corresponding to individual non-nutritive sucking events. These readings were processed further to calculate an average value for an infant in an environment in which external stimuli remained relatively unchanged; and an average value in an environment in which external stimuli were changed in a controlled fashion (in this case, by introducing an aroma proximate to the infant). This work is discussed in more detail below, but it is highlighted now to provide an example of how one version of an information device is used to quantify and process non-nutritive sucking events and rhythmic sucking patterns, which can provide indicia of an infant's perception of an aroma, or a change to an aroma.

Many different information devices may be used with the present invention. In addition to a desktop computer or a device for recording and/or displaying readings corresponding to non-nutritive sucking events (e.g., a multimeter displaying millivolt readings), one could use a personal-digital assistant, hand-held computer, a portable computer, or other compact device to receive, store, process, display, and/or transmit information corresponding to non-nutritive sucking events produced by an infant. Alternatively, a chart recorder or other such device for recording the detected non-nutritive sucking events may be used. As noted above, the information device may comprise a storage device, including, for example, RAM (i.e., Random Access Memory), ROM (i.e., Read-Only Memory), EPROM (i.e., Erasable Programmable Read-Only Memory), PROM (i.e., Programmable Read-Only Memory), RFID (i.e., Radio Frequency IDentification), or the like. Furthermore, information devices comprising storage devices such as those identified in the preceding list may be compact enough to be attached to the sensor used to detect non-nutritive sucking events produced by an infant. For example, an RFID device could be incorporated into a pacifier such that the device recorded the non-nutritive sucking events produced by an infant sucking on the nipple of the pacifier. When desired, an external device could be used to read the stored information on the RFID element. Alternatively, the information on the RFID element could be viewed on a display, either on the pacifier itself, or on a computer or other information device remote from the pacifier. In some versions of the invention, the information device will comprise only a storage device.

In some versions of the invention, a display connected to the sensor itself (e.g. a pacifier having both a pressure transducer to sense the non-nutritive sucking events and an information device comprising a display) could provide an indication or information corresponding to non-nutritive sucking events detected by the sensor and/or stored on the information device. In some examples of the invention, the display could be graphical in nature, with displayed images corresponding to a psychological state or state of well being indicated by non-nutritive sucking events and/or rhythmic sucking patterns. So, for example, if certain rhythmic sucking patterns were generally determined to correspond to a more stressful condition for an infant, then the graphical display on the pacifier might, for example, be an image of a face with a frown. Alternatively, if certain rhythmic sucking patterns were generally determined to correspond to a less stressful condition for the infant, then the graphical display on the pacifier might, for example, be an image of a face with a smile. Furthermore, color might be used, either alone, or in conjunction with graphical images, to convey information regarding the well being, stress level, psychological state, or perceptions of the infant. Any hardware and software capable of presenting a display of information, graphical or otherwise, might be used, including, for example, liquid-crystal displays, light-emitting diodes, and the like. In some versions of the invention, the graphical display is associated with an information device remote from the sensor used to detect non-nutritive sucking events and/or rhythmic sucking patterns (as is discussed in more detail in the following paragraph).

It should be noted that the information device need only be operatively connected to the sensor used to detect non-nutritive sucking events. Accordingly, the information device might be attached to the sensor itself. Or the information device might be at a location remote from the sensor, with information conveyed by an appropriate wavelength in the electromagnetic spectrum (e.g., radio waves); a conductive wire; or some mechanical connection (e.g., a pneumatic or hydraulic connection). In some cases, the information device may comprise one or more components attached to the sensor used to detect non-nutritive sucking events, and one or more components at a location remote from the sensor.

### Representative Approaches to Detecting the Presence of Aroma

As discussed above, the present invention is generally directed to systems, devices, and methods for detecting non-nutritive sucking events produced by an infant. One aspect of the present invention is to concurrently detect a change to an aroma proximate to the infant. Some versions of the invention involve the transport of a liquid to help effect a change to an aroma proximate to an infant. For example, a liquid having one or more volatile components, with these volatile components corresponding to an aroma, might be transported to a wick or other material, typically having a relatively high surface area, thereby helping to promote volatilization. Or liquid might be delivered to a nozzle, atomizer, or other such device that helps disperse the liquid, or component thereof, for the purpose of effecting a change in aroma (perhaps through a ventilation system or other ducts). In those instances where the release or change to the presence of an aroma relates to the flow or availability of liquid, then various properties associated with such flow or availability can be monitored (e.g., volumetric flow rate; revolutions per minute of a peristaltic pump head, with the revolutions per minute correlated with a volumetric or mass flow rate; mass flow rate; mass of liquid in a reservoir, with the decrease in mass corresponding to the volatilization of the liquid and any chemical components corresponding to the aroma being released; and the like).

In some instances there would be no liquid flow, or at least no pumping of a liquid. Instead, at a given time, a device would be actuated, thereby effecting the release of volatile components that correspond to an aroma to be evaluated. For example, some devices release an aroma after a plastic container is opened, allowing for the release of aromatic compounds. Or a device is activated by heat or electric current, resulting in the release of aromatic compounds due to the input of energy. Also, even more simply, a source of an aroma may be placed in a location near the infant and any changes to non-nutritive sucking events and/or corresponding rhythmic patterns noted or monitored.

Of course such physical placement of a source by, for example, a nurse, will, concurrent to the infant's detection of the aroma, potentially result in other sensory stimuli to the infant (e.g., sounds, sights, smells, and perhaps touch associated with the nurse placing the aroma source near the infant). One approach to minimizing such stimuli is to remotely activate release of an aroma such that all stimuli other than the release of the aroma are minimized. This might be accomplished, for example, by having an aroma source that is activated remotely by a person turning on a switch that allows electric current to flow to the source, with the current, perhaps, generating heat that then volatilizes chemical components corresponding to the aroma. Or, alternatively, by the current actuating a mechanical or other switch that opens a port allowing chemical components to escape into the surrounding environment and be available for detection by the infant. Or by the current actuating a pump that delivers a liquid carrying one or more components capable of volatilizing, perhaps with the liquid being delivered to an atomizer, spraying unit, or other such device for dispersing the liquid or a component thereof. In another approach, the evaluation may be conducted while the infant is sleeping.

In some cases the mass of a solid or liquid comprising the aromatic compounds would be monitored. The change in mass would correspond to the amount of material, whether solid or liquid, that is volatilizing.

Alternatively, devices for measuring the concentration of volatilized materials in the air proximate to the infant would be measured. For example, air samples may be obtained and analyzed using appropriate analytical instruments, such as a chromatograph.

One version of a system of the present invention will generally have a sensor for detecting non-nutritive sucking events and/or rhythmic sucking patterns; a sensor for detecting a property that correlates with a change to an aroma; and one or more information devices for receiving, storing, displaying, processing, and/or transmitting information corresponding to the detected non-nutritive sucking events and detected property.

### Representative Uses of System of Present Invention

Information acquired using a system of the present invention may be used for a variety of purposes. By obtaining information pertaining to the well being, stress level, or other such indicator of an infant's perception of an aroma, a person or company can elect to develop and transmit messages and/or communications to consumers based on said information. For example, a message could be developed pertaining to comparing indicia of an infant's perception of one aroma to an infant's perception of a second aroma (or the infant's perception of the presence or absence of an aroma; or the infant's perception of different concentrations or intensities corresponding to a given aroma; and the like). If differences between these aromas, their presence or absence, or their intensity/concentration levels result in differences in characteristics of individual non-nutritive sucking events or rhythmic sucking patterns (e.g., average amplitude), then a communication to consumers (e.g., caregivers of infants) based on this information could be fashioned. The communication or message could take the form of a newspaper advertisement, a television advertisement, a radio or other audio advertisement, items mailed directly to addressees, items emailed to addresses, Internet Web pages or other such postings, free standing inserts, coupons, various promotions (e.g., trade promotions), co-promotions with other companies, copy and the like, boxes and packages containing the product, and other such forms of disseminating information to consumers or potential consumers.

Systems of the present invention may be used to evaluate the effect of deodorizers or other materials that help reduce or eliminate aromas (e.g., activated carbon). So, for example, if an infant's non-nutritive sucking events and/or corresponding rhythmic sucking pattern changed concurrent to the introduction of a deodorizer or other material proximate to the infant such that the intensity or concentration of an aroma systematically decreased, then the sponsor of the evaluation would have evidence supporting the conclusion that the infant perceived the effect of the deodorizer on the aroma.

In some cases the system may be used to ensure that an article of manufacture employs an aroma that is pleasing to a caregiver, but is not perceived as unduly stressful to the infant. Thus if an article of manufacture employing an aroma at a given intensity was deemed pleasing to adult participants in a use test (i.e., as to the aroma), and if an infant's non-nutritive sucking events and/or corresponding rhythmic sucking pattern did not change as a result of introducing the same aroma, at that same intensity, to a location proximate to the infant, then the sponsor of the evaluation would have evidence supporting the conclusion that the infant did not detect the presence of the aroma, and yet the aroma was perceived as pleasing by adult participants in the use test.

The above approach could also be used when evaluating an improvement or change to an existing product. For example, a communication or message could be developed pertaining to comparing indicia of an infant's perception of one product employing an aroma to an infant's perception of a product employing a second aroma (or one product with an aroma, or without an aroma; or one product with different intensity/concentration levels of a given aroma; and the like). If differences in characteristics of individual non-nutritive sucking events or rhythmic sucking patterns (e.g., average amplitude) provide indicia of an infant (or, more likely, populations of infants) perceiving this difference between the two products (whether having different aromas, or with and without an aroma, or at different concentration/intensity levels of a given aroma, and the like), then a communication to consumers based on this information could be fashioned and disseminated as discussed above.

A system of the present invention could also be used to formulate, modify, or terminate a research-and-development project based on the acquired information. If, for example, infants sensing an aroma (whether alone or deployed in a product) provide indicia of an improved state of well being, or lower stress level, compared to other infants (e.g., infants part of a control group not exposed to the same aroma), then a company could elect to invest in a new research-and-development program in which the modification was further developed.

A system of the present invention could be used to evaluate a plurality of design variables for a product. For example, a product, such as a stuffed toy, could be designed to have a scent or aroma adapted to calm or have some other desired effect promoting an infant's well being. A system of the present invention can be used to evaluate: the identity and amount of ingredients that might be employed to effect an aroma; and other variables relating to design of a product (color, texture, sound, etc.). A product designer or researcher can use experimental designs to determine the effects of each of the design variables of interest. The product designer or researcher would look at the effect of changes to these independent design variables on the dependent variable of interest, in this case the characteristics of individual non-nutritive sucking events or rhythmic sucking patterns (e.g., average amplitude).

Of course the preceding paragraphs provide examples of measurements, tests, use tests, and other ways by which the performance of an aroma, or product employing an aroma, can be characterized. It should be understood that the present invention may be used to provide indicia of an infant's perception of the performance of a variety of contexts in which one or more aromas are deployed.

### EXAMPLES

### Example 1

Construction of One Version of a Device Adapted to Detect Non-nutritive Sucking Events and/or Rhythmic Sucking Patterns. A DISNEY brand pacifier, manufactured by The First Years, One Kiddie Drive, Avon, Mass., was obtained.

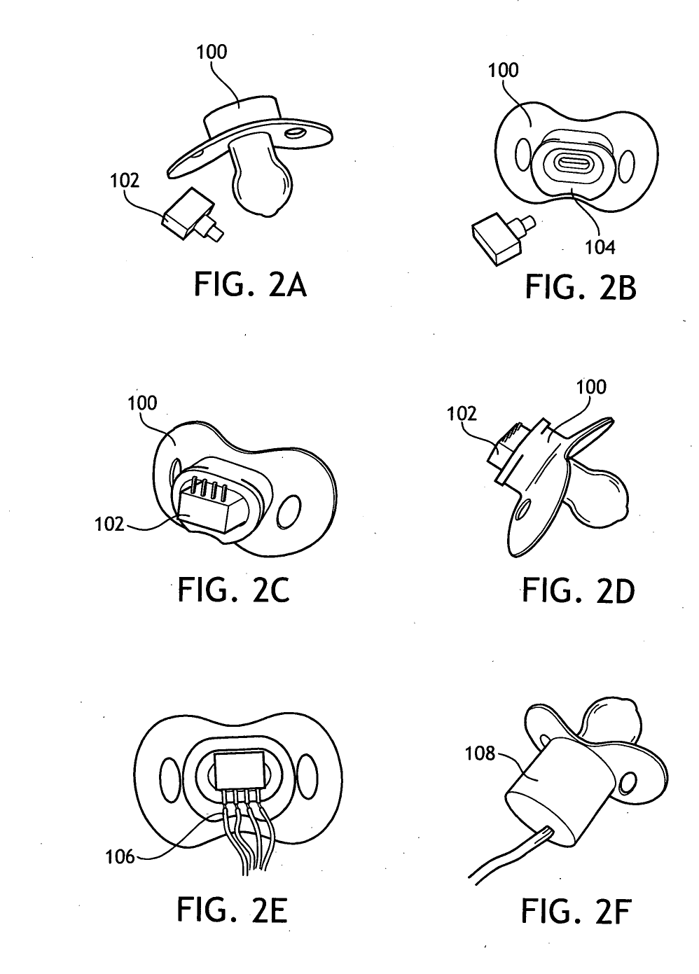

The pacifier was modified as depicted in FIGS. 2A through 2F. FIG. 2A depicts the pacifier 100 along with a pressure transducer 102 obtained from Omega Engineering, having offices at One Omega Drive, Box 4047, Stamford, Conn. This particular pressure transducer (model no. PX26-001GV) was adapted to detect pressure readings from zero to one pounds per square inch. FIG. 2B depicts the pacifier 100 modified such that the back of the base has been removed to reveal the end of the nipple 104 which is attached to the base.

FIGS. 2C and 2D show the pressure transducer 102 inserted into the opening created by removing the back of the base. In this version of a device adapted to detect non-nutritive sucking events and/or rhythmic sucking patterns, epoxy is used to attach the pressure transducer 102 to the base of the pacifier. A sufficient amount of epoxy was used so that epoxy filled the space between the portion of the transducer that was inserted into the base and the inner wall of the base. I.e., a substantially air-tight seal was formed between the transducer and the base of the pacifier.

FIG. 2E shows a four-conductor ribbon cable 106 soldered to the pressure transducer. FIG. 2F depicts a protective sleeve 108 positioned around the pressure transducer.

Note also that a drill with a #60 drill bit was used to drill a hole through the solidified epoxy (at a location near the transducer) such that a port to the open end of the nipple was created. The port allowed the nipple to vent air from its interior when squeezed or compressed; and to draw air into its interior when allowed to return to its original shape. The port allowed the nipple to collapse and return to its original shape during use. The port was added because the original, unmodified pacifier had a vent/port, but the original vent was eliminated when we removed the back of the pacifier and attached the transducer with epoxy.

### Example 2

Construction of Another Version of a Device Adapted to Detect Non-nutritive Sucking Events and/or Rhythmic Sucking Patterns. A MAM brand pacifier made by MAM Babyartikel GEsmbh, Lorenz-Mandl-Gasse 50, 1160 Wien, Austria, was obtained.

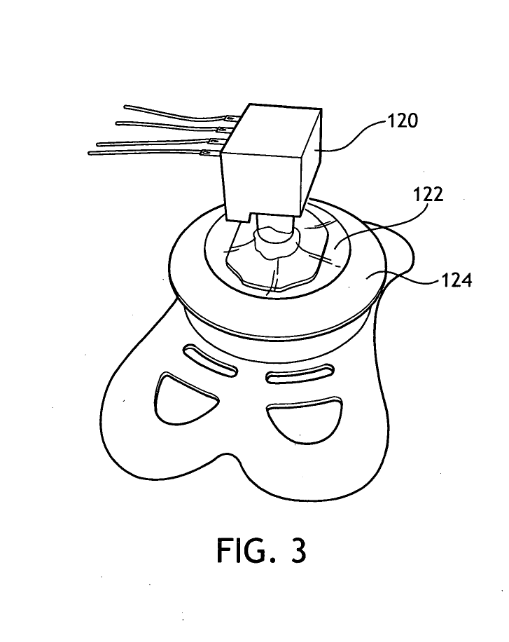

The pacifier was modified as follows. As depicted in FIG. 3, a pressure transducer 120, obtained from Omega Engineering, having offices at One Omega Drive, Box 4047, Stamford, Conn., was attached to the open end 122 of a base 124 of a pacifier. Epoxy was used to attach the pressure transducer to the pacifier. A sufficient amount of epoxy was used so that epoxy filled the space between the portion of the transducer that was inserted into the base and the inner wall of the base. I.e., a substantially air-tight seal was formed between the transducer and the base of the pacifier. This particular pressure transducer was adapted to detect pressure readings of zero to 5 pounds per square inch.

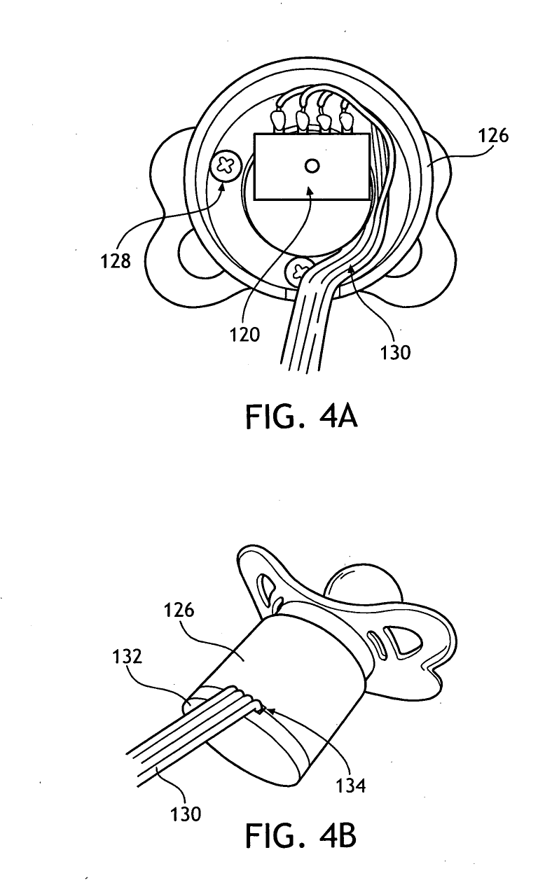

FIG. 4A depicts a protective cup 126 attached to the base of the pacifier using #2-56 screws 128. A four-conductor ribbon cable 130 was soldered to each of the four connectors of the pressure transducer. FIG. 4B shows the modified pacifier with an end cap 132 attached to the protective cup 126. A notch 134 allows for egress of the ribbon cable 130 from the interior of the protective cup 126.

Note also that a drill with a #60 drill bit was used to drill a hole through the solidified epoxy (at a location near the transducer) such that a port to the open end of the nipple was created. The port allowed the nipple to vent air from its interior when squeezed or compressed; and to draw air into its interior when allowed to return to its original shape. The port allowed the nipple to collapse and return to its original shape during use. The port was added because the original, unmodified pacifier had a vent/port, but the original vent was eliminated when we removed the back of the pacifier and attached the transducer with epoxy.

### Example 3

Construction of Another Version of a Device Adapted to Detect Non-nutritive Sucking Events and/or Rhythmic Sucking Patterns. A NUK brand pacifier, manufactured by NUK, MAPA GmbH, Industriestrasse 21-25, D-27404, Zeven, Germany, was obtained.

The pacifier was modified in the same way as the pacifier discussed in Example 2. In this case, however, a pressure transducer capable of detecting 0 to 15 pounds per square inch (0 to 100 millivolt DC, output) was attached to the base of the pacifier. The pressure transducer (model no. PX26-015GV; 0-15 psi) was obtained from Omega Engineering, having offices at One Omega Drive, Box 4047, Stamford, Conn.

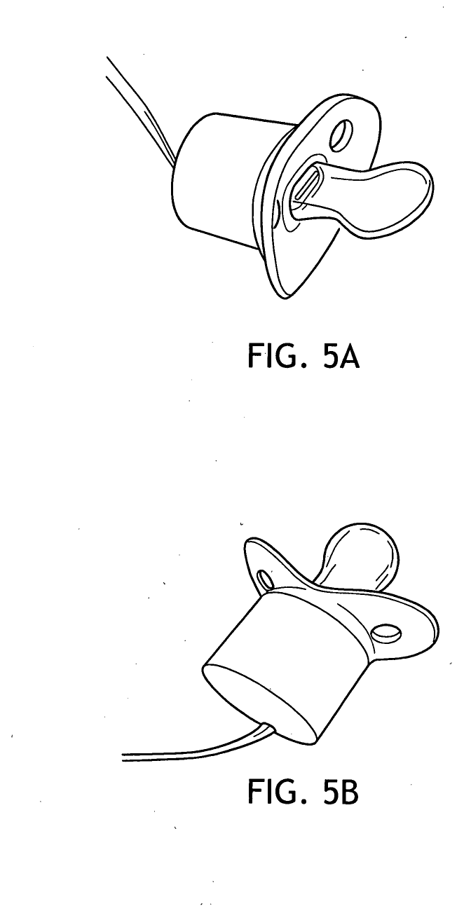

FIGS. 5A and 5B depict two different views of this version of a device adapted to detect non-nutritive sucking events and/or rhythmic sucking patterns.

### Example 4

### Construction of Another Version of a Device Adapted to Detect Non-Nutritive Sucking Events and/or Rhythmic Sucking Patterns

Several pacifiers were modified to include: a battery; a voltage regulator (model number MIC5219, from Micrel, a business having an office in San Jose, Calif.); a pressure transducer (model number 1451-015 G-T, from MSI Sensors, a business having an office in Hampton, Va.); a radio/microcontroller module (in this case one employing a ZIGBEE brand standard wireless protocol for transmitting or receiving data in digital form) (model number RC2200AT-SPPIO, from Radiocrafts, a business having an office in Sandakerveien 64, NO-0484 Oslo, Norway); an instrumentation amplifier (model number AD627, from Analog Devices, a business having an office in Norwood, Mass.); and an ultra-precision operational amplifier (model number OP177, from Analog Devices, a business having an office in Norwood, Mass.).

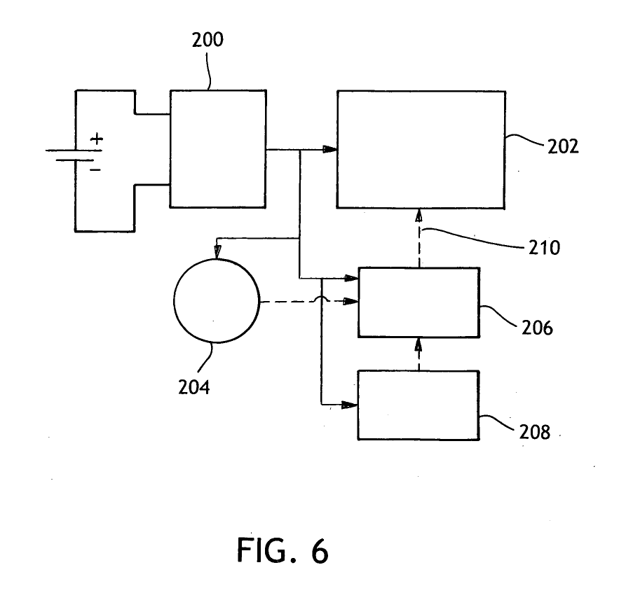

In this representative embodiment, the wireless pacifier was powered by the output of a 3-volt, 500 milli-ampere voltage regulator 200 in FIG. 6. Typically, two 3-volt coin cells configured in series provided the raw unregulated voltage. This was necessary since the regulator needed greater than 3.1 volts to operate. The 3-volt coin cells arranged in series (for a total of 6 volts), as compared to a single 3-volt coin cell, improved the utilization of available batteries.

As noted above, in this representative embodiment of a wireless pacifier, the main component was a ZIGBEE brand ready-radio module, designated as 202 in FIG. 6. This module contained the necessary components for bi-directional wireless communications. The module also contained a microcontroller employing a 10-bit analog-to-digital converter input. The radio and microcontroller worked together to process information and create a wireless serial link between the pacifier and, as discussed below, a computer. The microcontroller engaged the radio at a rate of 10 Hertz to transmit 80 bytes of information, each cycle, for a transmission rate of 800 bytes per second (i.e., a 100 Hertz sampling rate). The 80 bytes of information consisted of 10 packets of 8 bytes each. The binary encoded data was transmitted in hex format (16-byte characters) as follows: byte 0: pacifier wireless module identification; byte 1-3: sample number, with each increment corresponding to 10 milliseconds in time; byte 4-7: pressure in IEEE 754 32-bit format; byte 8: pacifier wireless module identification; byte 9-11: sample number, with each increment corresponding to 10 milliseconds in time; byte 12-15: pressure in IEEE754 32 bit format.

The pressure transducer 204, which in this representative embodiment was capable of detecting in the range of 0 to 15 gauge pounds per square inch, was of a piezo-resistive silicon type. The transducer employed a bridge network that required voltage excitement. The differential pressure output signal was passed to an instrumentation amplifier 206. The instrumentation amplifier was provided a reference voltage from an ultra-precision amplifier 208 which was configured as a voltage follower fed by a simple voltage divider. The instrumentation amplifier then provided the proper level signal to the module's analog-to-digital converter 210 where its output is adjusted to represent the actual output of the pressure transducer in pounds per square inch, or “PSI.”

The wireless pacifier was prepared much like the wired-in pacifiers described in the previous examples, with the exception that the above components, interconnected as depicted in FIG. 6, were placed in the base of the pacifier. Thus the resulting, modified pacifiers looked generally like that depicted in FIGS. 5A and 5B with the exception, of course, of a wire protruding from the base of the pacifier (because the pacifiers transmitted data in wireless fashion). The pacifiers modified as generally described above included two DISNEY brand pacifiers, one for ages zero and over, and one for ages 3 months and over, both of which had a silicon nipple; two MAM brand pacifiers, one designated as “Crystal” for ages 6 months and over, and one designated “Pearl” for ages zero to 6 months, both of which had a silicon nipple; two NUK brand pacifiers, one designated for ages zero to 6 months, and one designated for 7 to 18 months, both of which had a latex nipple; and two PLAYTEX brand pacifiers, one designated for ages 3 months and over and having a silicon nipple, and one for the same age range but having a latex nipple.

### Example 5

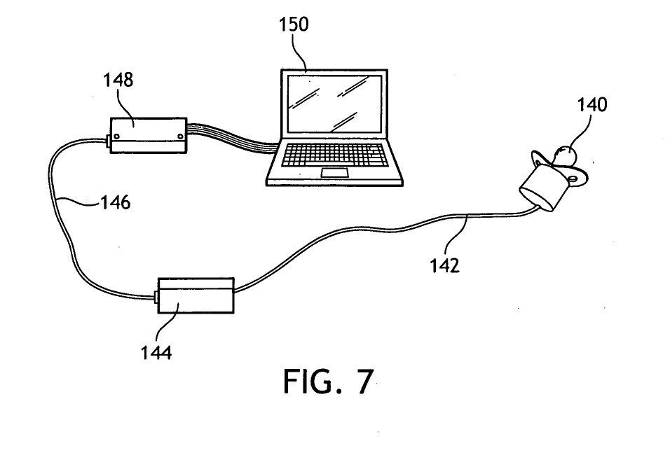

One Version of a System for Evaluating an Aroma, or Product Effecting an Aroma. Proximate to a User of the Product. First, a device for detecting non-nutritive sucking events and/or rhythmic sucking patterns was made in accordance with the description in Example 2 above. This device 140 was then connected to other equipment as depicted in FIG. 7. Four wires 142 exit the device (here, a modified pacifier). Two of these four wires were connected to a pair of ten-volt, direct-current batteries. The batteries were used to excite the transducer, and were positioned in a battery holder 144 available from Keystone Electronics Corp., a business having offices in Astoria, N.Y. The two remaining wires 146, which were associated with a 0 to 50 millivolt analog output from the pressure transducer, were connected to an analog-to-digital interface 148, in this case model number NI DAQCard-6036E (for PCMCIA; 200 kS/s, 16-bit, 16 Analog input multifunction DAQ), available from National Instruments Corp, a business having offices at North Mopac Expressway, Austin, Tex. It should be noted that other signal outputs, including analog signal outputs from pressure transducers, may be used, including, for example, AC voltage, DC voltage, AC current, or DC current. In addition, while not done here, the output signal may be amplified using appropriate electronic devices to boost the voltage or current to a higher value which is directly proportional to the pressure applied. Still another possible adaptation would be to utilize an apparatus commonly know as a voltage- or current-to-air pressure transducer (i.e. “I/P”, or “V/P” transducer) to change a pressure transducer's analog voltage or current output signal to a mechanical signal such as a pneumatic air pressure output or a hydraulic pressure output in proportion to the analog signal input. For example, a 4-20 milliamp DC signal output from a pressure transducer can produce a proportional 3-15 PSI pneumatic signal output to enable or facilitate a mechanical motion (e.g., to perform a physical tack, or tasks).

The analog-to-digital interface does what its name states: it converts analog signals into a digital format that can be processed by, for example, a computer. The bit-rate conversion of the interface can be selected to provide adequate conversion of the signal from analog to digital. As noted with the selected interface identified above, we selected an interface with the capability of 16 bits per channel. The sampling frequency may be selected to accurately capture the frequency of the individual non-nutritive sucking events, and associated rhythmic sucking patterns, being produced. Generally, choosing a sampling frequency that is twice that of the frequency of the event (Nyquist criterion) will present a lower range for sampling frequency. Other sampling frequencies include 100 times the Nyquist criterion or 1000 times the Nyquist criterion (e.g., 1000 Hertz). Additionally, to reduce our requirements for handling large amounts of data (e.g., when displaying the data visually), we at times filtered, i.e., reduced, the data to a frequency of 4 Hertz.

Here the analog-to-digital interface was connected to a Compaq EVO NG10C computer (designated by the number 150 in FIG. 7). LabView software, version 6.1, was obtained from National Instruments. LabView software configures a computer to acquire, store, process, and display data transmitted to the computer from various sources, in this case digital values transmitted from the analog-to-digital converter. In this case, the software was used to process digital information corresponding to the analog signals, in millivolts, effected by an infant sucking on a modified pacifier equipped to detect non-nutritive sucking events.

### Example 6

### One Version of a System for Evaluating an Aroma, or Product Effecting an Aroma, Proximate to a User of the Product

The representative system is much like that in the previous example, but which operates wirelessly. Accordingly, a pacifier like that described in Example 4 is employed, which, as noted above, wirelessly transmits data, including pressure, to a computer. The receiver for the wireless-pacifier system is a Chipcon (headquarter in Oslo, Norway) model CC2420 DB ZIGBEE demonstration board. This board is connected to a computer (identified below) through a RS-232 interface which uses 9-pin D-Subminiature connectors and was powered by an AC adapter. As the demonstration board receives packets of information, it relays this information back to the computer through the RS-232 connection at a baud rate of 57.6K. The computer used was a Hewlett-Packard nc6220, and LabView 8.2 software was used to collect the data from the RS-232 interface, display, and store the data.

### Example 7

### Test Showing Change in Rhythmic Sucking Pattern in Response to Change to an Aroma

At a clinical research facility, a caregiver, in this case the infant's parent, carried a 3-month-old male infant into a room. The infant remained in a car seat/portable carrier for the evaluation. The seat was placed on a table at one end of the room, with the seat positioned so that the infant's field of view was directed toward a nearby wall. The infant was in a reclined position, with the infant's head higher than his feet. A computer for monitoring non-nutritive sucking events and corresponding rhythmic sucking patterns was located at the other end of the room. During the test the infant could not see any activity by a researcher, or the equipment used to monitor data transmitted by a wireless pacifier. The caregiver remained in a position such that the infant saw the caregiver, at least while the infant remained awake.

The infant was given one of the pacifiers described in Example 4 above. This particular infant was given a NUK brand pacifier, one which was, according to the caregiver, the same as the typical unmodified pacifier used by the infant at home. After the caregiver gave the infant the wireless pacifier, the infant began sucking on the pacifier, thereby producing non-nutritive sucking events. The pressure transducer inside the pacifier detected pressure changes corresponding to these sucking events, with the pressure, in pounds per square inch, being transmitted to a computer (along with other information, identified above in Example 4). The computer was configured, using appropriate software—in this case LabView software, version 8.2, from National Instruments—to acquire, store, process, and display data being transmitted from the wireless pacifier (see Example 6 for additional information on the equipment used in this evaluation). In this case the data was displayed on an x-y plot, with the y dimension corresponding to pressure in pounds per square inch, and the x dimension corresponding to time.

First non-nutritive sucking events effected by the infant were monitored to obtain a baseline for future comparisons (i.e., comparisons between the pattern of non-nutritive sucking events obtained in the absence of a controlled external stimulus and the pattern of non-nutritive sucking events obtained in the presence of a controlled external stimulus). As stated above, the caregiver/mother of the infant was visible to the infant at all times. After the infant entered a light sleep (as evidenced by the infant closing his eyes), a baseline value corresponding to an average pressure was obtained. I.e., after one or two minutes the pattern of individual non-nutritive sucking events was stable and regular, and had the appearance of a repeating wave-form pattern (with the amplitude of each wave corresponding to a pressure increase, followed by a decrease, due to the infant periodically sucking on the pacifier). As discussed earlier, each suck flexes the nipple of the pacifier, causing a pressure change inside the nipple. The sensor selected for this exemplary system, a pressure transducer, detects these pressure changes.

After having established a baseline rhythmic sucking pattern, and with the infant still asleep—as evidenced, in part, by his eyes being closed, a nurse removed a fragranced blotter strip from a wrapping and placed the strip directly under the child's nose for approximately 10 seconds. After this ten-second interval, the nurse removed the fragrance blotter from under the infant's nose and returned the blotter to its sealed wrapping.

The information device used to monitor the non-nutritive sucking events employed a graphical user interface in which the sucking events were displayed, as described above, as a function of time, concurrent to the infant participant's exposure to the aroma source. The researcher monitoring the information device, in this case a computer, marked the time at which the fragranced blotter strip was placed beneath the infant's nose, and the time at which the fragranced blotter strip was removed (an elapsed time, as noted above, corresponding to ten seconds). Any changes to the non-nutritive sucking events, and the corresponding rhythmic sucking pattern, were monitored concurrent to, and after, the infant was exposed to the aroma.

The resulting data was then analyzed. Analysis of the data showed a measurable increase in average pressure from 0.6 pounds per square inch prior to the infant's exposure to the aroma, to an average pressure of 1 pound per square inch after the aroma was presented for ten seconds at the high-intensity level. The aroma or fragrance generally corresponded to that which an adult might describe as characterizing the scent typical of a baby powder. For this example, “high intensity” refers to those samples, and the corresponding aroma, in which blotter paper was dipped into the fragrance oil and removed so that the blotter paper was substantially saturated with the oil. Excess liquid was allowed to drain from the blotter paper. Adult observers noted that the scent corresponding to the high-intensity blotter paper/aroma was “very strong.”

The experiment was repeated with an aroma at a low intensity. No change to pressure was observed. In this case, “low intensity” refers to those samples, and corresponding aroma, in which a 10 micro-liter aliquot of the aforementioned oil was deposited on the blotter paper using a pipette.

### Prophetic Example 8

Use of System for Evaluatinq an Aroma to Help Substantiate an Advertising Claim or Message. A system, like the exemplary system discussed in Examples 4 and 6 above, is used to evaluate different aromas with a number of infant participants. The study helps establish both an average amplitude or pressure for a baseline rhythmic sucking pattern (i.e., before an infant participant is exposed to an aroma) and an average amplitude or pressure for rhythmic sucking patterns corresponding to the presence, absence, and/or intensity of one or more aromas. If detected differences are deemed different using an appropriate statistical or other basis, and this difference is attributable, in whole or in part, to the presence, absence and/or intensity of one or more aromas, then the resulting data may be used to help substantiate a possible advertising claim or message regarding the aroma as perceived by the tested infants. For example, if one tested aroma corresponded to an average pressure greater than the baseline pressure, then this data might be used to support an advertising claim based on this difference.

Other approaches may be used for the above purpose. For example, rather than evaluate the average amplitude or pressure of a selected collection of individual non-nutritive sucking events, the frequency of said events might be evaluated. Alternatively, specific patterns of such events might be used to compare aromas.

### Prophetic Example 9

Use of System for Evaluating an Aroma to Change or Initiate a Research-and-Development Project or Program. A system, like the exemplary system discussed in Examples 4 and 6 above, is used to evaluate different aromas with a number of infant participants. The study helps establish both an average amplitude or pressure for a baseline rhythmic sucking pattern (i.e., before an infant participant is exposed to an aroma) and an average amplitude or pressure for rhythmic sucking patterns corresponding to the presence, absence, and/or intensity of one or more aromas. If detected differences are deemed different using an appropriate statistical or other basis, and this difference is attributable, in whole or in part, to the presence, absence and/or intensity of one or more aromas, then the resulting data may be used to help support a decision to change, terminate, or initiate a research-and-development project directed to the aroma, or a product employing the aroma. For example, if one tested aroma corresponded to an average pressure greater than the baseline pressure, then this data might be used to support a research-and-development project directed to making and selling the aroma, or products employing the aroma, to caregivers of infants or other purchasers, or potential purchasers.

Other approaches may be used for the above purpose. For example, rather than evaluate the average amplitude or pressure of a selected collection of individual non-nutritive sucking events, the frequency of said events might be evaluated. Alternatively, specific patterns of such events might be used to compare aromas.

### Prophetic Example 10

Use of System for Evaluating an Aroma to Identify Ranges of Values Corresponding to Rhythmic Sucking Patterns Indicative of States of Well Being of Infants. A system, like the exemplary system discussed in Examples 4 and 6 above, is used to evaluate a number of infants to determine both an average amplitude or pressure for a baseline rhythmic sucking pattern and average amplitudes or pressures for rhythmic sucking patterns corresponding to presence, absence and/or intensity of an aroma. A sufficient number of infants are tested such that ranges of amplitudes/pressures, frequencies, or other characteristics of rhythmic sucking patterns are identified that correspond to specific states of well being or stress.

We claim:

1. A system for evaluating an infant's perception of an aroma, the system comprising:
a sensor adapted to detect non-nutritive sucking events produced by an infant;
a source comprising an aroma; and
an information device operatively connected to the sensor, said information device adapted to accomplish one or more of receiving, storing, processing, displaying, or transmitting information corresponding to non-nutritive sucking events detected by the sensor.

2. The system of claim 1 wherein the sensor is a pressure transducer adapted to detect the non-nutritive sucking events.

3. The system of claim 2 wherein the pressure transducer is attached to a pacifier.

4. The system of claim 1 wherein the information device is attached to a pacifier.

5. The system of claim 1 wherein the information device is also operatively connected to the source, said information device further adapted to accomplish one or more of receiving, storing, processing, displaying, or transmitting information corresponding to the source's release of the aroma.

6. The system of claim 1 further comprising a second information device that is operatively connected to the source, said second information device adapted to accomplish one or more of receiving, storing, processing, displaying, or transmitting information corresponding to the source's release of the aroma.

7. The system of claim 1 wherein the information device is operatively connected to the sensor by conductive wire, a pneumatic connection, a hydraulic connection, a connection using a portion of the electromagnetic spectrum, or some combination of these.

8. A method for evaluating an infant's response to a first aroma, the method comprising the steps of:
(a) detecting non-nutritive sucking events produced by an infant; and
(b) relating the detected non-nutritive sucking events to a change in a first aroma proximate to the infant.

9. The method of claim 8 further comprising:
repeating steps 8(a) and 8(b) for a second aroma; and
comparing the detected non-nutritive sucking events produced by an infant proximate to the change in the first aroma to the detected non-nutritive sucking events produced by an infant proximate to the change in the second aroma.

10. The method of claim 8 further comprising communicating a message based on said relationship.

11. The method of claim 9 further comprising communicating a message based on said comparison.

12. A message adapted to be communicated, wherein the message comprises information produced using the system of claim 1.

13. A message referring to an aroma and adapted to be communicated, wherein substantiation of the message is based on non-nutritive sucking events.

14. A research-and-development project directed to selecting an aroma, wherein said research-and-development effort comprises an evaluation of non-nutritive sucking events.

15. A system for evaluating an infant's perception of an aroma, the system comprising:
a first sensor adapted to detect non-nutritive sucking events produced by an infant;
a second sensor adapted to detect a property characterizing the presence of an aroma proximate to the infant; and
an information device operatively connected to the first sensor, said information device adapted to accomplish one or more of receiving, storing, processing, displaying, or transmitting information corresponding to the non-nutritive sucking events detected by the first sensor.

16. A method for evaluating an infant's perception of an aroma, the method comprising the steps of:
(a) detecting non-nutritive sucking events produced by an infant proximate to a change to an aroma;
(b) characterizing the change; and
(c) relating the detected non-nutritive sucking events to the characterized change to the aroma.

17. The method of claim 16 wherein the non-nutritive sucking events are detected, at least in part, while the infant is in an environment substantially devoid of sounds.

18. The method of claim 17 wherein the non-nutritive sucking events are detected, at least in part, while the infant is located in a substantially acoustically isolated environment.

19. The method of claim 16 wherein the non-nutritive sucking events are detected using a pacifier that wirelessly transmits data corresponding to the non-nutritive sucking events to a remote information device.

20. The method of claim 16 wherein the non-nutritive sucking events are detected using a pacifier model selected from a plurality of pacifier models available for conducting the evaluation.

21. The method of claim 16 wherein the non-nutritive sucking events are detected, at least in part, while the infant is sleeping.

20. A method for evaluating an infant's perception of a product attribute, the method comprising the steps of:
(a) providing a plurality of pacifier models adapted to detect non-nutritive sucking events and wirelessly transmit data corresponding to the detected non-nutritive sucking events to a remote information device;
(b) selecting one of the pacifier models for the evaluation;
(c) using the selected pacifier model to detect non-nutritive sucking events produced by an infant who is exposed to a sensory stimulus that corresponds to the product attribute;
(d) relating the detected non-nutritive sucking events to the sensory stimulus, the product attribute, or both.

21. The method of claim 20 wherein the non-nutritive sucking events are detected, at least in part, while the infant is in an environment substantially devoid of sounds.

22. The method of claim 21 wherein the non-nutritive sucking events are detected, at least in part, while the infant is located in a substantially acoustically isolated environment.

23. The method of claim 20 wherein the pacifier model selected for the evaluation is chosen by the infant's caregiver.

24. The method of claim 20 wherein the pacifier model is selected to substantially match a pacifier model typically used by the infant.

25. The method of claim 20 wherein the non-nutritive sucking events are detected, at least in part, while the infant is sleeping.

26. The method of claim 20 wherein the remote information device displays data corresponding to the non-nutritive sucking events detected by the selected pacifier model.

27. The method of claim 20 wherein the remote information device employs a graphical user interface adapted to display time; the amplitude and/or the frequency of the detected non-nutritive sucking events; data corresponding to the identity and/or other characteristics of the infant participating in the evaluation; and data corresponding to the sensory stimulus and/or product attribute being evaluated.
